(19) United States
(12) Patent Application Publication
(10) Pub. No.: US 2023/0301930 A1
(43) Pub. Date: Sep. 28, 2023

(54) NANOPARTICLE CANCER THERAPY

(71) Applicant: UNIVERSITY OF SOUTH AUSTRALIA, Adelaide (AU)

(72) Inventors: Ivan Mark KEMPSON, Inglewood (AU); Tyron James TURNBULL, Parafield Gardens (AU)

(73) Assignee: UNIVERSITY OF SOUTH AUSTRALIA, Adelaide (AU)

(21) Appl. No.: 18/114,409

(22) Filed: Feb. 27, 2023

Related U.S. Application Data
(63) Continuation of application No. 16/758,792, filed on Apr. 23, 2020, now abandoned.

(30) Foreign Application Priority Data
Oct. 26, 2017 (AU) 2017904336

(51) Int. Cl. A61K 9/51; A61K 33/242; A61K 41/00; A61P 35/00; B82Y 40/00; B82Y 5/00

(52) U.S. Cl. CPC A61K 9/5123; A61K 33/242; A61K 41/0038; A61P 35/00; B82Y 5/00; B82Y 40/00

(57) ABSTRACT

Methods of potentiating chemotherapy or radiotherapy are disclosed. The methods comprise administering to a subject in need of chemotherapeutic or radiotherapeutic treatment an effective amount of a composition comprising biocompatible nanoparticles under conditions in which the nanoparticles alter one or more cell regulatory mechanisms in cells in which the nanoparticles are localised or other cells. Then one or more doses of a chemotherapeutic or radiotherapeutic treatment are administered to the subject either concurrently with or after the nanoparticles have altered the one or more cell regulatory mechanisms in the cells in which the nanoparticles are localised or other cells. Also disclosed are methods of enhancing the effects of chemotherapy or radiotherapy on a cell population, methods of increasing the amount of strand breaks in DNA in a cell, and methods of inducing cancer cell death.

### CROSS REFERENCE TO RELATED APPLICATIONS

This application is a continuation of U.S. Application No. 16/758,792, filed on April, 23, 2020, which is a 371 of PCT/AU2018/000205, filed on Oct. 26, 2018, which claims priority from Australian Provisional Patent Application No. 2017904336, filed on 26 Oct. 2017, each of which is hereby incorporated by reference in its entirety.

### TECHNICAL FIELD

The present disclosure relates to the use of nanoparticles in cancer therapy.

### BACKGROUND

Radiation treatment (“radiotherapy”) is used in approximately 60% of cancer treatments and its use contributes to approximately 40% of cures yet it accounts for only 5-10% of cancer related treatment costs. With unsustainable increases in health care, radiotherapy can play a key role in effective cancer treatment whilst keeping health care costs down. Radiotherapy has been improved by hardware development (e.g. intensity modulate radiotherapy) in shaping radiation dose to the tumour volume. Unfortunately these hardware developments have plateaued and radiotherapy is limited by the spatial quality and precision of dose delivery. This has resulted in many cancers having very limited improvement in mortality rates since approximately 2006.

Radiation results in breaks in one or both strands of the DNA molecules inside cells. Cells in all phases of the cell cycle are susceptible to the effects of radiation but DNA damage in cancer cells is more likely to be lethal because these cells are less capable of repairing their DNA.

As with any cancer treatment, specificity of the treatment regime to the cancer cells to be treated is important and side effects of treatment arise as a result of damage to healthy cells and tissue. Recurrence of tumours after radiotherapy has been partly attributed to the presence of radioresistant hypoxic cells and/or cells in their S-phase. Increased radiation doses are therefore required to damage the radioresistant cancer cells. However, this leads to an increased risk of damage to normal, healthy tissue. Attempts to date to improve radiotherapy regimes have involved increasing the dose of radiation delivered to the tumour while minimizing radiation to healthy tissue, sensitizing radio-resistant cancer cells to conventional doses of radiation, and targeting cancer cells specifically while administering radiation.

In recent years, intravenously administered nanoparticles have been explored as potential anti-cancer agents. These nanoparticles accumulate preferentially within tumours largely as a result of their size and passive extravasation from the leaky, chaotic and immature vasculature of tumours. Interaction of nanoparticles of high atomic weight elements (“high Z elements”) with incident radiation can be used to provide a localised dose enhancement and the selectivity of the nanoparticle for the target cells allows the radiation dose to be enhanced at the target.

To date, the enhancement of radiation doses using high Z element nanoparticles has been attributed to a photoelectric effect mechanism whereby the incident energy is absorbed by an electron within the element and the electron ejected from its orbit. If this electron is an inner-shell electron, the hole left behind by its ejection is filled by electrons that drop down from outer orbits—the resulting transition in binding energies of that electron result in the release of characteristic X-rays that are unique to the metal being irradiated (Hainfeld et al. 2004). For example, published international patent application WO 2012048099 A2 (Osiris Therapeutics, Inc.) discloses that gold nanoparticle-loaded cells are able to interact with electromagnetic radiation or magnetic fields and states that “interaction of nanoparticles with electromagnetic radiation or magnetic fields enhances energy deposition to local environments”. Alternatively, or in addition, nanoparticle radiosensitization may enhance the generation of reactive oxygen species and subsequent damage to DNA to lead to cell death. However, no model of nanoparticle sensitization has been able to adequately explain cell radiobiological response.

There is a need for an improved understanding of the mechanisms associated with cell radiobiological responses and for improved radiotherapy treatments based on the improved understanding.

### SUMMARY

In a first aspect, provided herein is a method of potentiating chemotherapy or radiotherapy, the method comprising:
administering to a subject in need of chemotherapeutic or radiotherapeutic treatment an effective amount of a composition comprising biocompatible nanoparticles under conditions in which the nanoparticles alter one or more cell regulatory mechanisms either in cells in which the nanoparticles are localised or other cells; and
administering one or more doses of a chemotherapeutic or radiotherapeutic treatment to the subject either concurrently with or after the nanoparticles have altered the one or more cell regulatory mechanisms.

In a second aspect, provided herein is a method of enhancing the effects of chemotherapy or radiotherapy on a cell population, the method comprising:
exposing the cell population to an effective amount of a nanoparticle composition comprising biocompatible nanoparticles under conditions in which at least some of the nanoparticles are localised in cells of the cell population to form nanoparticle-laden cells and the localised nanoparticles alter one or more cell regulatory mechanisms in either the nanoparticle-laden cells or other cells; and
exposing the cell population to a chemotherapeutic agent or ionizing radiotherapy concurrently with or after the nanoparticles have altered the one or more cell regulatory mechanisms in the nanoparticle-laden cells or other cells.

In a third aspect, provided herein is a method of increasing the amount of strand breaks in DNA in a cell, the method comprising:
exposing the cell to an effective amount of a nanoparticle composition comprising biocompatible nanoparticles under conditions in which at least some of the nanoparticles are localised in the cell to form a nanoparticle-laden cell and the localised nanoparticles alter one or more cell regulatory mechanisms in the nanoparticle-laden cell.

In certain embodiments of the third aspect, the method further comprises exposing the nanoparticle-laden cell to a chemotherapeutic agent or ionizing radiotherapy concurrently with or after the nanoparticles have altered the one or more cell regulatory mechanisms in the nanoparticle-laden cells or other cells.

In a fourth aspect, provided herein is a method of inducing cancer cell death, the method comprising:
exposing cancer cells to be treated to an effective amount of a nanoparticle composition comprising biocompatible nanoparticles under conditions in which at least some of the nanoparticles are localised in the cancer cells to form nanoparticle-laden cancer cells and the localised nanoparticles alter one or more cell regulatory mechanisms in either the nanoparticle-laden cancer cells or other cells; and
exposing the nanoparticle-laden cancer cells or other cells to a chemotherapeutic agent or ionizing radiotherapy concurrently with or after the nanoparticles have altered the one or more cell regulatory mechanisms in the nanoparticle-laden cancer cells or other cells under conditions to cause cancer cell death.

In a fifth aspect, provided herein is a chemotherapeutic or radiotherapeutic treatment method comprising:
administering to a subject in need of chemotherapeutic or radiotherapeutic treatment an effective amount of a nanoparticle composition comprising biocompatible nanoparticles; and
administering one or more doses of a chemotherapeutic agent or ionizing radiotherapy to the subject either concurrently with or after administration of the nanoparticle composition;
wherein the nanoparticle composition is administered under conditions in which the nanoparticles alter one or more cell regulatory mechanisms in cells in which the nanoparticles are localised or other cells, and one or more doses of a chemotherapeutic agent or ionizing radiotherapy are administered to the subject either concurrently with or after the nanoparticles have altered the one or more cell regulatory mechanisms in cells in which the nanoparticles are localised or other cells.

In some embodiments of the first to fifth aspects, the biocompatible nanoparticles comprise a material selected from one or more of the group consisting of: gold, iron, carbon, boron, silica, magnesium, titanium, titania, manganese, arsenic, silver, platinum, palladium, tin, tantalum, ytterbium, zirconium, hafnium, terbium, thulium, cerium, dysprosium, erbium, europium, holmium, lanthanum, neodymium, praseodymium, lutetium, copper, strontium, samarium, radium, gadolinium, iodine, molybdenum, technetium, thallium, rubidium, phosphorous, actinium, bismuth, actinium, fluorine, gallium, krypton, xenon, rubidium, yttrium, chromium, cobalt, rhenium, mixtures of any of the aforementioned materials, salts of any of the aforementioned materials, compounds containing any of the aforementioned materials, and complexes containing any of the aforementioned materials.

In some embodiments of the first to fifth aspects, the biocompatible nanoparticles comprise a gold material. In certain embodiments, the gold material is gold metal. In certain embodiments, the gold material is coated gold nanoparticles. The coated gold nanoparticles may have a coating selected from any one or more of a silica coating and an organic coating.

In some embodiments of the first to fifth aspects, the biocompatible nanoparticles comprise boron nitride.

### BRIEF DESCRIPTION OF DRAWINGS

Embodiments of the present disclosure will be discussed with reference to the accompanying drawings wherein:

FIG. 1 shows a cross correlative image set produced after irradiation with a clinical X-ray source;

FIG. 2 shows an example of a zoomed in region of cells after irradiation with 4 Gy. Cell nuclei are shown in blue and DNA DSBs in green. The adjacent histogram shows the distribution of DNA DSBs in a cell population and a fit with a ‘normal’ distribution equation;

FIG. 3 shows data for the nanoparticle content for three different cancer cell lines;

FIG. 4 shows a plot for PC-3 cancer cells exposed to 4 Gy from a clinical X-ray source on number of DNA breaks (foci) and amount of gold nanoparticles;

FIG. 5 is a plot showing that above ~15 pg of Au the nanoparticles cause an impairment in the repair of DNA. In the plot shown, the impairment in DNA repair is significant to the p<0.05 level at content greater than ~20pg;

FIG. 6 shows the division of cells into sub-populations based on their growth phase;

FIG. 7 shows that cell repair mechanisms have an important impact on cellular sensitivity to radiation repair;

FIG. 8 shows that nanoparticles have specific effects on cells in different phases and that the nanoparticle uptake probabilities are comparable for cells co-cultured with nanoparticles for a time of 2 hrs, or proportionally equivalent to ~10% of the cells’ doubling time;

FIG. 9 shows that three sub-populations are indistinguishable with regard to the cumulative probability of nanoparticle uptake;

FIG. 10 shows that the sensitivity to radiation by way of ability to repair DNA varies within a specific growth phase;

FIG. 11 shows that the nanoparticles have least impact, by way of DNA DSB repair as a function of nanoparticle content (represented by the slope of the line fitting the data), on the most radiation sensitive cells (G2 and M phase);

FIG. 12 shows the ability to impair DNA DSB repair varies through the cell cycle according the genetic state of the cells;

FIG. 13 is a plot of normalised DAPI intensity against cell count for control cells showing the number of cells in each cell growth phase;

FIG. 14 is a plot of normalised DAPI intensity against cell count for cells exposed to 5 nm gold nanoparticles showing the number of cells in each cell growth phase;

FIG. 15 is a plot of normalised DAPI intensity against cell count for cells exposed to 10 nm gold nanoparticles showing the number of cells in each cell growth phase;

FIG. 16 shows the mean TMS pixel intensity for the cells shown in FIGS. 13 to 15 in the G1 phase;

FIG. 17 is a plot of the mean TMS pixel intensity against density for the cells shown in FIGS. 13 to 15 in the G1 phase;

FIG. 18 shows the mean TMS pixel intensity for the cells shown in FIGS. 13 to 15 in the S phase;

FIG. 19 is a plot of the mean TMS pixel intensity against density for the cells shown in FIGS. 13 to 15 in the S phase;

FIG. 20 shows the mean TMS pixel intensity for the cells shown in FIGS. 13 to 15 in the G2 phase; and

FIG. 21 is a plot of the mean TMS pixel intensity against density for the cells shown in FIGS. 13 to 15 in the G2 phase.

### DESCRIPTION OF EMBODIMENTS

The present disclosure results from the inventors’ findings that a better chemotherapeutic and/or radiotherapeutic response can be achieved clinically by using nanoparticles to alter cell regulatory mechanisms, such as gene expression, in cancer cells. The altered cell regulatory mechanisms then interfere with DNA damage repair mechanisms and render the cells vulnerable to chemotherapeutic agents used in chemotherapeutic treatment and/or to ionizing radiation used in radiotherapeutic treatment.

The inventors’ findings indicate that the nanoparticles are not interacting with radiation as is the case with some prior art techniques such as the one disclosed in WO 2012048099 A2. Rather, the nanoparticles are acting as a DNA damage response inhibitor which, in turn, renders cells more susceptible to chemotherapeutic and/or radiotherapeutic treatments.

Provided herein is a method of potentiating chemotherapy or radiotherapy. The method comprises administering to a subject in need of chemotherapeutic or radiotherapeutic treatment an effective amount of a nanoparticle composition comprising biocompatible nanoparticles under conditions in which the nanoparticles alter gene expression in cells in which the nanoparticles are localised or in other cells. One or more doses of a chemotherapeutic or radiotherapeutic treatment is administered to the subject either concurrently with or after the nanoparticles have altered the one or more cell regulatory mechanisms.

As used herein, the term “other” cells refers to cells surrounding the cells in which the nanoparticles are localised. The other cells may be in physiological communication with the adjacent nanoparticle-laden cells. Without intending to be bound by any specific theory, it is possible that the nanoparticle-laden cells may communicate with other cells and potentiate the effects of chemotherapy or radiotherapy in the other cells.

As discussed previously, it is generally considered that X-ray photons interact with nanoparticles to enhance the effects of radiotherapy in the treatment of cancer. Currently the mechanism(s) of the enhanced effects are not known, however it is widely accepted that they are based on the physical interaction of the photon and the nanoparticle. As such, physical interactions of the X-rays with the nanoparticles are generally thought to enhance the radiation dose deposited in cells. However, the work described herein shows that the dominant mechanism is due to a biological response of cells to nanoparticles, rather than due to the X-ray interaction with the nanoparticle. The data presented herein suggests that nanoparticles instigate changes in the production of enzymes, other proteins and biomolecules inside a cell that act in inhibiting DNA repair after irradiation or treatment of a chemotherapeutic agent.

The methods described herein may therefore provide a benefit of an improved effect of radiotherapeutic or chemotherapeutic treatments by potentiating those treatments. This may, for example, lead to improved toxicity profiles for existing or new radiotherapeutic or chemotherapeutic treatments.

Also provided herein is a method of potentiating chemotherapy or radiotherapy. The method comprises administering to a subject in need of chemotherapeutic or radiotherapeutic treatment an effective amount of a nanoparticle composition comprising biocompatible nanoparticles under conditions in which the nanoparticles alter gene expression in cells in which the nanoparticles are localised or other cells. One or more doses of a chemotherapeutic or radiotherapeutic treatment is administered to the subject either concurrently with or after the nanoparticles have altered the one or more cell regulatory mechanisms in the cells in which the nanoparticles are localised or other cells.

The methods described herein can be used to potentiate chemotherapy and/or radiotherapy. As used herein, the term “potentiating” when used in relation to chemotherapeutic or radiotherapeutic treatment means increasing the effectiveness of one or more chemotherapeutic agents or increasing the effectiveness of radiation treatment or therapy for the treatment of cancer in a subject. A determination as to whether a chemotherapeutic treatment has been potentiated or is of increased effectiveness can be made by detecting an improvement in the anti-cancer activity of a specified dosage regimen of a chemotherapeutic agent when administered following, or concurrently with, an effective amount of the nanoparticle composition as compared to administration of the same dosage of chemotherapeutic agent without the nanoparticle composition. An increased effectiveness of radiation therapy in conjunction with treatment with the nanoparticle composition can be determined by substantially the same method. The term “increase”, and any grammatical variants of that term, refer to an increase in the specified parameter of at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300% or more.

A subject in need of chemotherapeutic or radiotherapeutic treatment may be a subject in need of cancer treatment. As used herein the term “cancer” refers to any benign or malignant abnormal growth of cells and includes lymphomas, carcinomas and sarcomas, and other neoplastic conditions, as these terms are commonly used in the art. Examples include, without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms’ tumour, cervical carcinoma, testicular carcinoma, bladder carcinoma, pancreatic carcinoma, stomach carcinoma, colon carcinoma, prostatic carcinoma, genitourinary carcinoma, thyroid carcinoma, oesophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukaemia, acute lymphocytic leukaemia, chronic lymphocytic leukaemia, acute myelogenous leukaemia, chronic myelogenous leukaemia, chronic granulocytic leukaemia, acute granulocytic leukaemia, hairy cell leukaemia, neuroblastoma, rhabdomyosarcoma, Kaposi’s sarcoma, polycythemia vera, essential thrombocytosis, Hodgkin’s disease, non-Hodgkin’s lymphoma, soft-tissue sarcoma, osteogenic sarcoma, primary macroglobulinemia, and retinoblastoma. In some embodiments, the cancer is selected from the group of tumour-forming cancers.

The chemotherapeutic treatment that is potentiated can be any suitable chemotherapy using one or more chemotherapeutic agents. A variety of chemotherapeutic agents are known for administration to patients in need of chemotherapy including, but not limited to: 1,3-bis(2-chloroethyl)-1-nitrosourea, bleomycin sulfate, 5-fluorouracil, 6-mercaptopurine, prednisone, methotrexate, lomustine, mitomycin, cisplatin, procarbazine hydrochloride, dacarbazine, cytarabine, streptozocin, epipodophyllotoxin, etoposide, taxol, anthracycline antibiotics such as doxorubicin hydrochloride (adriamycin) and mitoxantrone, vinca alkaloids such as vinblastine sulfate and vincristine sulfate, and alkylating agents such as meclorethamine, cyclophosphamide and ifosfamide. These agents are typically used alone or in combination chemotherapy for the treatment of neoplastic diseases, as described in The Merck Manual, 19th Ed., R.S. Porter, ed., Merck Sharp & Dohme Corp. (Whitehouse Station, N.J. 2011).

Subjects can be administered an effective amount of a chemotherapeutic agent in a dosage form, at a dosage rate and for a dosage period that can be determined by a clinician based on factors including the subject’s weight, the nature of the chemotherapeutic agent, etc. Administration of the chemotherapeutic agent can be intravenous, parenteral, subcutaneous, intramuscular, or any other acceptable systemic method. The formulations of pharmaceutical compositions contemplated by the above dosage forms can be prepared with conventional pharmaceutically acceptable excipients and additives, using conventional techniques, such as those described in Remington: The Science and Practice of Pharmacy, 22^{nd} Ed., Lloyd V. Allen, ed., Pharmaceutical Press, 2013.

The radiotherapeutic treatment that is potentiated can be any suitable radiotherapy that instigates DNA damage, such as X-rays, electrons, protons, neutrons, hadrons, and other ions. Methods for the treatment of cancer and/or tumours using radiation therapy are well known in the art. See, e.g. The Merck Manual, 19th Ed., supra. Contemplated radiation sources for use in radiotherapy include: X-ray sources, neutron sources, gamma ray sources, nuclear particle sources, ion sources, electron sources, proton sources, microwave sources, beta particle sources, alpha particle sources, visible light sources, infrared sources, ultraviolet sources and radio frequency sources. Radiation sources, as used herein, also include radioactive isotopes.

Administration of the radiotherapeutic treatment can be by any of the methods known in the art. Ionising radiation or other radiation leading to the generation of reactive species can be applied to a target volume including a cancerous tumour and surrounding tissue. Radiation may also be applied to other areas of the body, such as draining lymph nodes involved with a tumour.

Before and/or during chemotherapeutic or radiotherapeutic treatment a subject is treated with an effective amount of a nanoparticle composition. The term “effective amount” as used herein means that the amount of nanoparticles contained in the composition administered is of sufficient quantity to achieve the intended purpose, such as, in this case, to perpetuate DNA Double Strand Breaks (DSBs) in one or more cells to be treated, such as cancer cells or tumour cells. The presence of DSBs in a cell of interest can be determined using one or more markers for DSBs, as is known in the art. Suitable markers include yH2AX, 53BP1, ATM, MDC1, RAD50, RAD51 and BRCA1. Alternatively stated, a “therapeutically effective amount” is an amount that will provide some alleviation, mitigation, or decrease in at least one clinical symptom in the subject (e.g. reduced tumour size, decreased incidence of metastasis, etc. for subjects having a form of cancer). The therapeutic effects need not be complete or curative, as long as some therapeutic benefit is provided to the subject.

The nanoparticle composition comprises biocompatible nanoparticles. As used herein, the term “nanoparticle”, and any grammatical variant thereof, refers to a particle that is about 0.1 nm to about 200 nm in diameter. In some embodiments, the nanoparticle has a diameter of from about 5 nm to about 100 nm or from about 5 nm to about 200 nm. In some embodiments, the particle or nanoparticle is about 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 975 or 999 nm in diameter.

The biocompatible nanoparticles may comprise a material selected from one or more of the group consisting of: gold, iron, carbon, boron, silica, magnesium, titanium, titania, manganese, arsenic, silver, platinum, palladium, tin, tantalum, ytterbium, zirconium, hafnium, terbium, thulium, cerium, dysprosium, erbium, europium, holmium, lanthanum, neodymium, praseodymium, lutetium, copper, strontium, samarium, radium, gadolinium, iodine, molybdenum, technetium, thallium, rubidium, phosphorous, actinium, bismuth, actinium, fluorine, gallium, krypton, xenon, rubidium, yttrium, chromium, cobalt, rhenium, mixtures of any of the aforementioned materials, salts of any of the aforementioned materials, compounds containing any of the aforementioned materials, and complexes containing any of the aforementioned materials.

The biocompatible nanoparticles may be coated. For example, the biocompatible nanoparticles may comprise a metal or metal oxide core and a silica coating. Silica coated biocompatible nanoparticles can be prepared by any suitable method. For example, silica coated biocompatible nanoparticles can be prepared by reacting a hydroxyl-functionalised silane with a nanoparticle in a substantially aqueous phase under conditions to induce silanization of the nanoparticle, as described in published international patent application No. WO2016013975 A1 (Agency For Science, Technology And Research) the details of which are hereby incorporated by reference.

In another example, the biocompatible nanoparticles may comprise a metal or metal oxide core and an organic coating. The organic coating comprises a monolayer or multilayers of organic compounds. The organic compounds may be small molecules, monomers, oligomers and/or polymers. The backbone of the organic compounds in the organic coating may comprise C₃-C₂₄ alkyl chains and a functional moiety such as a thiol, a thiolate, a sulfide, a disulfide, a sulfite, a sulfate, a carbamate, an amine, a phosphine, a carboxylate, a cyanate, or an isocyanate moiety. Nanoparticles comprising a metal or metal oxide core and an organic coating can be prepared by any suitable method, such as the method described in U.S. Pat. 8903661 B2 (Technion Research And Development Foundation Ltd.), for example.

In order to increase accumulation of the nanoparticles in a tumour or cancer cell(s), “stealth” agents may be used to reduce the immunogenicity of the nanoparticles. For example, nanoparticles may be, optionally, coated with a lipid or phospholipid. The lipid or phospholipid can be any of the numerous lipids that contain a diglyceride, a phosphate group, and a simple organic molecule such as choline. Examples of phospholipids include, but are not limited to, phosphatidic acid (phosphatidate) (PA), phosphatidylethanolamine (cephalin) (PE), phosphatidylcholine (lecithin) (PC), phosphatidylserine (PS), and phosphoinositides which include, but are not limited to, phosphatidylinositol (PI), phosphatidylinositol phosphate (PIP), phosphatidylinositol bisphosphate (PIP2) and phosphatidylinositol triphosphate (PIP3). Additional examples of PC include DDPC, DLPC, DMPC, DPPC, DSPC, DOPC, POPC, DRPC, and DEPC as defined in the art. Phospholipids or lipids used to coat the nanoparticles can be functionalised with various agents, such as polyethylene glycol (PEG) to form pegylated lipids or pegylated phospholipids.

The nanoparticles may also be “targeted” using a ligand that will bind to the surface of the target cell. For example, targeting agents can be covalently attached to functionalised lipids and/or phospholipids (e.g. pegylated lipids and/or phospholipids) to facilitate targeting of the nanoparticles to a specific cell (e.g. a cancer cell).

In some embodiments the biocompatible nanoparticles comprise a gold material. The gold material may be gold metal nanoparticles or coated gold nanoparticles.

In some embodiments, the biocompatible nanoparticles comprise an iron (Fe) material. In certain embodiments, the iron material is iron metal. For example, the biocompatible nanoparticles may comprise iron metal and/or iron oxide. For example, suitable iron nanoparticles can be prepared by the method of Huang et al.

In some embodiments, the biocompatible nanoparticles comprise carbon. In some embodiments, the biocompatible nanoparticles comprise boron. In some embodiments, the biocompatible nanoparticles comprise boron nitride. In some embodiments, the biocompatible nanoparticles comprise silica. In some embodiments, the biocompatible nanoparticles comprise magnesium oxide. In some embodiments, the biocompatible nanoparticles comprise titanium. In some embodiments, the biocompatible nanoparticles comprise titania. In some embodiments, the biocompatible nanoparticles comprise manganese. In some embodiments, the biocompatible nanoparticles comprise arsenic. In some embodiments, the biocompatible nanoparticles comprise iron-platinum. In some embodiments, the biocompatible nanoparticles comprise barium sulfate. Iron-platinum, manganese and barium sulfate biocompatible nanoparticles can also be used for properties for image-guided radiation therapy with MRI contrast. Others may provide contrast in X-ray computed tomography for image-guided radiation therapy.

In some embodiments, the nanoparticle composition may optionally contain one or more additional radiosensitisers. Complexes containing platinum, ruthenium, palladium, iron, cobalt, nickel, copper, rhodium, gold, silver and boron can be used as radiosensitisers. Some non-limiting examples of radiosensitisers include the platinum complexes cisplatin, oxaliplatin and carboplatin.

In addition to the biocompatible nanoparticles, the nanoparticle composition may contain one or more pharmaceutically acceptable carriers, adjuvants, excipients or diluents. As used herein, “pharmaceutically acceptable” means that the material is suitable for administration to a subject and will allow desired treatment to be carried out without giving rise to unduly deleterious side effects. As used herein, the term “pharmaceutically acceptable carrier” refers to any suitable pharmaceutical diluent and/or excipient, such as phosphate buffered saline and/or isotonic saline solution. Examples of pharmaceutically acceptable carriers, diluents and excipients may be found, for example, in Remington: The Science and Practice of Pharmacy 22nd Ed., supra.

The nanoparticle composition may also contain various other materials, such as pH adjusting and/or buffering agents, tonicity adjusting and/or buffering agents and lipid-protective agents (e.g. agents that that protect lipids against free-radical damage, such as alpha-tocopherol). The nanoparticle composition may be formulated so as to be suitable for administration via any known method, including, but not limited to, oral, intravenous, subcutaneous, intramuscular, intrathecal, intraperitoneal, intra-arterial, intratumoral, intrarectal, intravaginal, intranasal, intragastric, intratracheal, sublingual, transcutaneous and intrapulmonary.

The subject can be any mammal, avian, reptile, amphibian or fish. Mammalian subjects may include, but are not limited to, humans, non-human primates (e.g. monkeys, chimpanzees, baboons, etc.), dogs, cats, mice, hamsters, rats, horses, cows, pigs, rabbits, sheep and goats. In particular embodiments, the subject is a laboratory animal. Human subjects may include neonates, infants, juveniles, adults, and geriatric subjects.

As used herein, the terms “treatment”, “treat” and “treating” refer to providing a subject with the nanoparticles disclosed herein in an effort to alleviate, mitigate, or decrease at least one clinical symptom in the subject.

Accumulation of the biocompatible nanoparticles in cancer cells is the result of the enhanced permeation and retention (EPR) effect due to the vascular leakage and abnormal vessel architecture of cancerous areas. Accumulation of the biocompatible nanoparticles in cancer cells may occur via transcellular transport (i.e. the transport of the nanoparticle into the tumour volume through cells) and/or paracellular transport (i.e. the transport of the nanoparticles into the tumour volume through tight junctions). In order to use the EPR effect for tumour accumulation, the nanoparticles must be within a size range to reduce extravasation into non-tumour areas but also allow accumulation through the EPR effect. In general, nanoparticles less than 5.5 nm in diameter (or its longest dimension) may be cleared from the blood through the kidneys, reducing their availability for accumulation in cancer cells. On the other hand, nanoparticles greater than 200-400 nm are unlikely to accumulate through the EPR because the nanoparticles exceed the size of the fenestrations in the tumour.

The nanoparticles alter one or more cell regulatory mechanisms in cells in which they are localised or other cells. The nanoparticles may alter gene expression in cells in which they are localised. For example, the nanoparticles may be responsible for or involved in the down regulation of, or interference with, genes for proteins, or the proteins themselves, involved in DNA repair and synthesis, or their respective substrates, such as ribonucleotide reductase and DNA polymerase; and enzymes involved in the catalysis of DNA nucleotides (dAMP, dGMP, dCMP and dTMP) such as thymidylate synthase and kinase; guanine monophosphate synthase (GMPS); inosine-5′-monophosphate dehydrogenase (IMPD); deoxycytidine kinase (dCK); uridine monophosphate kinase (UMPK) and their respective substrates; and genes or proteins involved with: Direct Repair (MGMT); Base Excision Repair (OGG1, UNG and XRCC1); Nucleotide Excision Repair (XPA, XPC, ERCC1, ERCC2, ERCC4, ERCC5, ERCC6 and XAB2); Double Strand Break Repair (XRCC2, XRCC3, XRCC4, XRCC5, BRCA1, BRCA2 and UBE2V2); Post-Replicative Repair (UBE2A, UBE2B and UBE2N); DNA replication (TYMS, RRM2B, RRM2, RRM1, TOP3A and TOP3B); and Telomere maintenance (TERT, TERF1 and TERF2).

For example, the nanoparticles may reduce the expression of thymidylate synthase which is a key enzyme that catalyses the conversion of deoxyuridine monophosphate (dUMP) to deoxythymidine monophosphate (dTMP). dTMP is an essential precursor for DNA biosynthesis. Reduced expression of thymidylate synthase then impairs the ability for the cells to recover, especially via the homologous recombination pathway, after receiving subsequent DNA damage by chemotherapy and/or radiotherapy. For example, following or during administration of the nanoparticle composition, a thymidylate synthase inhibitor chemotherapeutic agent may be administered. 5-Fluorouracil is a thymidylate synthase inhibitor in clinical use. It is widely used for the treatment of colorectal, pancreatic, breast, head and neck, gastric, and ovarian cancers. Raltitrexed is a folate analogue that is approved as first-line therapy for advanced colorectal cancer in Europe, Australia, Canada, and Japan. Pemetrexed is an antifolate analogue that has shown promising activity in several solid tumour types, including mesothelioma. ZD9331 has shown activity in patients with refractory ovarian and colorectal cancer. Capecitabine is an oral fluoropyrimidine carbamate that was designed to generate 5-FU preferentially in tumour cells.

In another example, the nanoparticles may reduce the expression of ribonucleotide reductase which is a key enzyme that catalyses the formation of deoxyribonucleotides from ribonucleotides. For example, following or during administration of the nanoparticle composition, a ribonucleotide reductase inhibitor chemotherapeutic agent may be administered. Examples of ribonucleotide reductase inhibitor chemotherapeutic agent include motexafin gadolinium, hydroxyurea, fludarabine, cladribine, gemcitabine, tezacitabine, triapine, gallium maltolate, and gallium nitrate.

The present inventors’ work has shown that the ability to impair DNA DSB repair varies through the cell cycle according the genetic state of the cells and that cells in the S-phase, which correlate with cancer therapy failure, are the cells most prone to nanoparticle induced disruption of DNA DSB repair.

One or more doses of the chemotherapeutic or radiotherapeutic treatment is/are administered to the subject either concurrently with or after administration of the nanoparticle composition.

Administration of the chemotherapeutic or radiotherapeutic treatment “concurrently with or after” means that the nanoparticle composition is administered either (a) prior to the start of the chemotherapeutic or radiotherapeutic treatment, (b) prior to the resumption of chemotherapeutic or radiotherapeutic treatment where said treatment has been stopped or suspended, or (c) during the course of chemotherapeutic or radiotherapeutic treatment, i.e. concurrently with administration of other chemotherapeutic agents or radiotherapy.

Also provided herein is a method of enhancing the effects of chemotherapy or radiotherapy on a cell population. The method comprises:
exposing the cell population to an effective amount of a nanoparticle composition comprising biocompatible nanoparticles under conditions in which at least some of the nanoparticles are localised in cells of the cell population to form nanoparticle-laden cells and the localised nanoparticles alter one or more cell regulatory mechanisms in the nanoparticle-laden cells or other cells; and
exposing the cell population to a chemotherapeutic agent or ionizing radiotherapy concurrently with or after the nanoparticles have altered the one or more cell regulatory mechanisms in the nanoparticle-laden cells or other cells.

Also provided herein is a method of increasing the amount of strand breaks in DNA in a cell. The method comprises:
exposing the cell to an effective amount of a nanoparticle composition comprising biocompatible nanoparticles under conditions in which at least some of the nanoparticles are localised in the cell to form a nanoparticle-laden cell and the localised nanoparticles alter one or more cell regulatory mechanisms in the nanoparticle-laden cell or other cells.

In certain embodiments of this aspect, the method further comprises exposing the nanoparticle-laden cell to a chemotherapeutic agent or ionizing radiotherapy concurrently with or after the nanoparticles have altered the one or more cell regulatory mechanisms in the nanoparticle-laden cells or other cells.

Also provided herein is a method of inducing cancer cell death. The method comprises:
exposing cancer cells to be treated to an effective amount of a nanoparticle composition comprising biocompatible nanoparticles under conditions in which at least some of the nanoparticles are localised in the cancer cells to form nanoparticle-laden cancer cells and the localised nanoparticles alter one or more cell regulatory mechanisms in the nanoparticle-laden cancer cells or other cells; and
exposing the nanoparticle-laden cancer cells or other cells to a chemotherapeutic agent or ionizing radiotherapy concurrently with or after the nanoparticles have altered the one or more cell regulatory mechanisms in the nanoparticle-laden cancer cells or other cells under conditions to cause cancer cell death.

Also provided herein is a chemotherapeutic or radiotherapeutic treatment method comprising:
administering to a subject in need of chemotherapeutic or radiotherapeutic treatment an effective amount of a nanoparticle composition comprising biocompatible nanoparticles; and
administering one or more doses of a chemotherapeutic agent or ionizing radiotherapy to the subject either concurrently with or after administration of the nanoparticle composition;
wherein the nanoparticle composition is administered under conditions in which the nanoparticles alter one or more cell regulatory mechanisms in cells in which the nanoparticles are localised and the one or more doses of a chemotherapeutic agent or ionizing radiotherapy are administered to the subject either concurrently with or after the nanoparticles have altered the one or more cell regulatory mechanisms in cells in which the nanoparticles are localised.

### EXAMPLES

### Example 1- Effect of Nanoparticles on DNA Double Strand Breaks (DSBs)

A gold nanoparticle (AuNP) solution (0.6 nM) was prepared by the standard sodium citrate reduction method proposed by Turkevich et al. AuNPs were first treated with a polyethylene glycol (PEG) solution consisting of a mix of short chain (458.6 Da) to long chain (2000 Da) PEG (Rapp Polymere) at a volume ratio of 4:1 based on a protocol established by Liu et al. The PEGylated AuNPs were then conjugated with human transferrin (Sigma Aldrich) after activation of terminal carboxylic acid groups using standard carbodiimide chemistry to increase cell uptake.

Seed particle size (14 nm) was confirmed with dynamic light scattering. Three measurements were taken with mean of the three measurements yielding a measured mean particle size of 14.26 nm. PEG and Transferrin conjugation was confirmed with UV-Vis measurements.

For the other TS measurements, PEG coated AuNPs were purchased from Jomar Life Research. Particles were 10 nm in diameter excluding the 2000 Da PEG coating as per supplier datasheets.

The PEG coated gold nanoparticles conjugated with transferrin were cultured at a concentration of 0.6 nM with prostate cancer (PC-3) cells. The human prostate cancer cell line, PC-3, was purchased from ECACC. Cells were cultured in RPMI-1640 culture media (Sigma-Aldrich); media was supplemented with 10% fetal bovine serum (Sigma-Aldrich), 2% penicillin/streptomycin (Sigma-Aldrich) and 1% L-Glutamine (Sigma-Aldrich). Cultures were grown in a humidified chamber at 37° C. with CO₂ levels maintained at 5%. Cells plated for experiments were at passage 9 and removed from tissue culture flasks at 80% confluence.

For γH2AX quantification cells were seeded at passage 9 and cultured on tissue culture treated polymer coverslips (Ibidi, Germany) at a density of 20,000 cells per well in removable silicon wells (Sarstedt, Germany). Cells were incubated in a humidified chamber at 37° C. in 5% CO₂ overnight to facilitate maximum cell adhesion after such the media was removed and replaced by serum free media containing the transferrin conjugated AuNPs at a AuNP concentration of 6 nM. Cells were incubated for 2 hours in the NP media after which the media was removed and replaced with fresh media and placed back in the incubator for a further 1 hour prior to transport for irradiation.

Cells were incubated for 1 hour post irradiation prior to fixation and staining for yH2AX foci. Cell nuclei were identified and imaged using DAPI. After images were acquired, samples where rinsed thoroughly with Milli-Q (MQ) water and dried in preparation for XRF analysis.

For measurement of TS protein expression, cells were plated in 6 well plates (Corning) at a density of 500,000 cells/well (passage 15). Following overnight adhesion cells were co-cultured with 10 nm PEG-AuNPs at a concentration of 10 µg/ml for 2 hours. After co-culture cells were washed, fixed and stained for TS expression quantify by imaging flow cytometry.

Cells were irradiated at the Royal Adelaide Hospital (RAH) Radiation Oncology Department with a microSelectron Iridium-192 source (Nucletron B-V., Veenendaal, the Netherlands) used for high dose rate brachytherapy treatments.

The radiation dose was delivered to the cells by sending the Iridium source to a known position using the departmental source calibration “jig”. The cells in the wells were positioned at a distance of 4 cm from the source position. An estimation of the irradiation time necessary to deliver 4.4 Gy to the cells was made using the current air kerma strength of the Ir-192 source and AAPM TG-43 formalism (Nath et al).

An estimation for the irradiation time can be obtained simply by applying an inverse square law correction to the air kerma strength at 1 m (assuming kerma is equal to dose in medium) and converting air kerma to dose in water at the cell layer:

$$\text{Dose} / \text{Kerma} \approx {S}_{k} \times {\left(\frac{100 \text{cm}}{\text{d}}\right)}^{2} \times T \times {\left(\frac{{μ}_{a b}}{ρ}\right)}_{w a t e r , a i r}$$

Where Sₖ is the air kerma strength, d is the distance from the source to the cells,

$${\left(\frac{{μ}_{a b}}{ρ}\right)}_{w a t e r , a i r}$$

is the ratio of the mass energy absorption coefficients for water to air and T is the irradiation time. The air kerma strength at the time of irradiation was 18.78 mGym²/h. The ratio of the mass energy absorption coefficients was taken to be 1.11², and assuming a mean photon energy of 300 keV for an Ir-192 source. The irradiation time required to deliver 4.4 Gy at a distance of 4 cm from the source using this method is 1224 seconds.

The dose delivered for this irradiation time was then calculated using AAPM TG-43 formalism (this is an approximation, as the protocol assumes the source is entirely within a water medium):

$${D}^{\&} \left(r , θ\right) = {S}_{k} \times \text{Λ} \times \frac{{G}_{P} \left(r , θ\right)}{{G}_{P} \left({r}_{0} , {θ}_{0}\right)} \times {g}_{L} \left(r\right) \times F \left(r , θ\right)$$

$${D}^{\&} \left(r , 90 °\right) = {S}_{k} \times \text{Λ} \times {r}^{- 2} \times {g}_{L} \left(r\right)$$

Where [symbol] (r,θ) is the dose rate at the point of interest, Sₖ is the air kerma strength, A is the dose rate constant, Gₚ is the point source approximation to the geometry function, g_{L} is the radial dose function, F is the anisotropy function, r is the distance from the source centre to the cells and θ is the angle between the axis of the source and the cells.

The anisotropy function reduces to unity under the conditions used to irradiate the cells. The dose rate constant was assumed to be 1.108 (based on “Dose Calculation for Photon-Emitting Brachytherapy Sources with Average Energy Higher than 50 keV: Full Report of the AAPM and ESTRO”) and a radial dose value of 1.004 was used at a distance of r=4 cm.

$${D}^{\&} \left(4 \text{cm} , 90 °\right) = 18788 \text{U} \times \text{1} \text{.108} \times {\text{4}}^{- 2} \times 1.004 \text{cGy} / \text{h}$$

For a treatment time of 1224 seconds

$$D \left(4 \text{cm} , 90 ° , 1224 \text{s}\right) = 4.4 \text{Gy}$$

This dose calculation was verified using Gafchromic EBT3 radiochromic film (International Specialty Products (ISP, Wayne, NJ)). A calibration curve for the EBT3 film was obtained by irradiating 4 calibration films using a 6 MV beam from a clinical Varian 600 C/D linear accelerator (Varian® Medical System, Palo Alto, CA) at the Royal Adelaide Hospital under reference dosimetry conditions. Doses of 0 (control), 1 Gy, 2 Gy and 4 Gy were delivered to the calibration films. A trial run was performed prior to cell irradiation to verify this method of dose calculation.

Once verified, thin sheets of EBT3 film with dimensions approximately (2.6 x 7.5 cm) were placed above and below the cell wells in order to estimate the delivered dose to the cells as a function of distance from the iridium source. The cells were irradiated for 1224 seconds with a 170.3 GBq source activity.

All films were analyses using Ashland Film QA Pro™ 3 software using a three-channel calibration curve. Sources of potential uncertainty in the delivered dose include: accurately estimating the distance of the source to the cells, correlating the position of the film with respect to the cell wells, lack of scatter medium (and thus lack of charged particle equilibrium) within the wells.

Post irradiation cells were fixed and stained for γH2AX foci to evaluate DNA DSB formation and DAPI for nuclei masking. Briefly, cells were washed with PBS and fixed 1 hr post irradiation with an ice cold solution consisting of 95% Ethanol (Chem-Supply) and 5% Acetic acid (Chem-Supply) for 10 mins. Following fixation cells were permeabilised for 15 mins using a PBS solution containing 0.5% Triton X-100 and then blocked using a buffer solution consisting of 5% Goat serum (Sigma-Aldrich) in PBS for 1 hr in a humidified incubator at 37° C. and 5% CO₂. After blocking cells were incubated for a further 1 hour in a humidified incubator at 37° C. and 5% CO₂ with 1/500 mouse anti-γH2AX (Millipore) antibody in PBS + 1% Goat serum. Fluorescent secondary antibody staining was performed by incubating the cells with Goat anti-mouse Alexa 488 (Abcam) at a 1/500 dilution in 1% Goat serum for 1 hr in the same conditions as the primary antibody step. Cells were then stained for nuclei identification and DNA content analysis using a DAPI solution (1 µg/ml) (Sigma-Aldrich) for 15 mins at room temperature. Finally cells were washed with MQ water for imaging.

Cells were fixed and stained for TS protein for analysis of TS expression via Imaging flow Cytometry. Briefly, cells were detached from the wells with trypsin (Sigma-Aldrich) which was then deactivated with RPMI. Cells were then concentrated via centrifugation and resuspended in ice cold PBS at a concentration of approximately 1-5 × 10⁶ cells/ml. Cells were fixed in 100 µL of formalin solution (Sigma-Aldrich) comprised of 10% formalin (approx. 4% formaldehyde). After further washing cells were permeabilised in a solution of 0.05% Triton X-100. Following permeabilisation cells were blocked for 30 mins with 5% BSA. The sample was then incubated with primary antibody (anti-Thymidylate synthase, rabbit polyclonal, Abcam) diluted in 1% BSA (1/1000) for 1 hour at 4° C. After further washing in PBS cells were incubated for 1 hour in the dark with secondary antibody (goat anti-rabbit IgG H&L (Alexa Fluor® 647) (Abcam), washed in PBS and stained with DAPI (1 µg/ml) (Sigma Aldrich) for cell nuclei identification.

Fluorescent images were acquired using a ZEISS LSM 710 laser scanning confocal microscope. (Carl Zeiss, Germany). A 20x objective was utilised with the 488 nm laser used for excitation of the yH2AX signal and 405 nm laser for the DAPI channel. Images dimensions were 7168 x 1024 pixels corresponding to approximate image size of 2.9 x 0.42 mm. These settings resulted in x and y resolutions of 0.415 µm. All images were acquired as z-stacks with a slice thickness of 2 µm and were 48 µm thick.

XRF elemental distributions were acquired at the Australian Synchrotron X-ray fluorescence microscopy beamline using methods described previously (Paterson et al; Turnbull et al 2015).

Cells stained for TS expression were imaged using an ImageStream^{x} Mark II multispectral imaging flow cytometer (AMNIS). Approximately 5,000 cells were analysed for each condition with cell images acquired at 40× magnification. Preliminary data analysis was performed to define individual cells using IDEAS image-analysis software (Version 6.2; AMNIS). Both control and treated data sets were merged for gating into relevant cell populations. Firstly, a single cell population was defined by excluding speed beads, cell doublet and triplets. This population was further sorted by selection of only DAPI positive cells for TS analysis. Once defined, population data was imported into MATLAB (2017a, Mathworks) for all further analysis. Compensation was performed to ensure accurate fluorescence intensity (a matrix was created based on single colour compensation files using the IDEAS software).

Maximum intensity projections of the raw confocal images were obtained using Image J software (National Institutes of Health, version 1.47t). Maximum projections were aligned and overlayed with the XRF elemental maps using Adobe Photoshop CC (2015 Adobe Systems Incorporated). Once aligned, the 3 layers (γH2AX, DAPI and Au) were exported as separate TIF files for quantification of γH2AX foci and Au content. Briefly, cell nuclei were identified by applying a minimum pixel intensity threshold to the DAPI channel along with in built MATLAB filters to define discrete cell nuclei. Following identification of the cell nuclei γH2AX foci were defined by grouping pixels of high intensity using a combination of thresholds, specifically, maximum and minimum pixel size of the groupings of pixels as well as a minimum pixel intensity requirement. We defined a foci as 4 connected pixels all with 125 or greater intensity in 8-bit scale. Lastly, the number of discrete foci present in each nuclei were counted and recorded for each cell. Along with these quantification steps, thresholds have been included within the analysis process to exclude misleading features, for example, clusters of cells being counted as a single cell. This quantification was performed with a custom script written in MATLAB 2017a. A more in-depth discussion of this script has been described previously (Turnbull et al 2017). All post image processing data analysis was performed in MATLAB (2017a, Mathworks).

DNA content was quantified by integrating the total DAPI pixel values through the Z-projection using custom analysis script in MATLAB (2017a, Mathworks).

One dimensional distributions were fitted with the inbuilt distribution fitting application in MATLAB. Fitting was described by equations for a probability distribution function:

$$P D F = \frac{1}{x σ \sqrt{2 π}} {e}^{- \frac{{\left(ln \left(x\right) - μ\right)}^{2}}{2 {σ}^{2}}}$$

Or, cumulative distribution function:

$$C D F = \frac{1}{2} + \frac{1}{2} \text{erf} \left[\frac{ln \left(x\right) - μ}{\sqrt{2 {σ}^{2}}}\right]$$

At each condition, the correlated data pairs, x = (x₁, x₂), were modeled using a bivariate normal (BVN) distribution

$$f \left(x\right) = \frac{1}{\left(2 π\right) {\left|\sum \text{}\right|}^{1 / 2}} exp \left(- \frac{{\left(x - μ\right)}^{′} \sum {}^{- 1} \left(x - μ\right)}{2}\right)$$

with mean vector, µ, and covariance matrix, ∑. A condition of the multivariate normal distribution is that the marginal distributions of the data be normally distributed. Confidence regions containing 1-α fraction of the probability of the BVN distribution are ellipsoids described by

$${\left(x - μ\right)}^{′} \sum {}^{- 1} \left(x - μ\right) = {χ}^{2} \left(α\right) .$$

For the BVN distribution, the conditional expectation of x₁ given x₂ is a line described by

$${E}_{\left({x}_{1}\right| {x}_{2}} = {μ}_{1} + ρ \frac{{σ}_{1}}{{σ}_{2}} \left({x}_{2} - {μ}_{2}\right)$$

Where ρ is the correlation coefficient between x₁ and x₂. Values of µ₁, µ₂, σ₁, and σ₂, the means and standard deviations of the marginal distributions, were estimated by fits of normal distribution to the 1-D data. The MATLAB built-in function corr was used to find ρ as well as to return a p value, testing the hypothesis of no correlation against the alternative that there is a non-zero correlation. If the p value is small, say less than 0.05, the correlation is defined as being significantly different from zero. The conditional expectation function is equivalent to a least squares fit of a linear function to the data.

All statistical analysis was performed with MATLAB (2017a, Mathworks). Choice on test was determined based on suitability of data. All t tests were 2 sided and multiple comparison corrections were applied as required. Significance was defined forp-values < 0.05 unless otherwise specified.

Custom scripts were utilised in this work to perform the multivariate analysis, quantification and cross-correlation of γH2AX foci and Au content per cell.

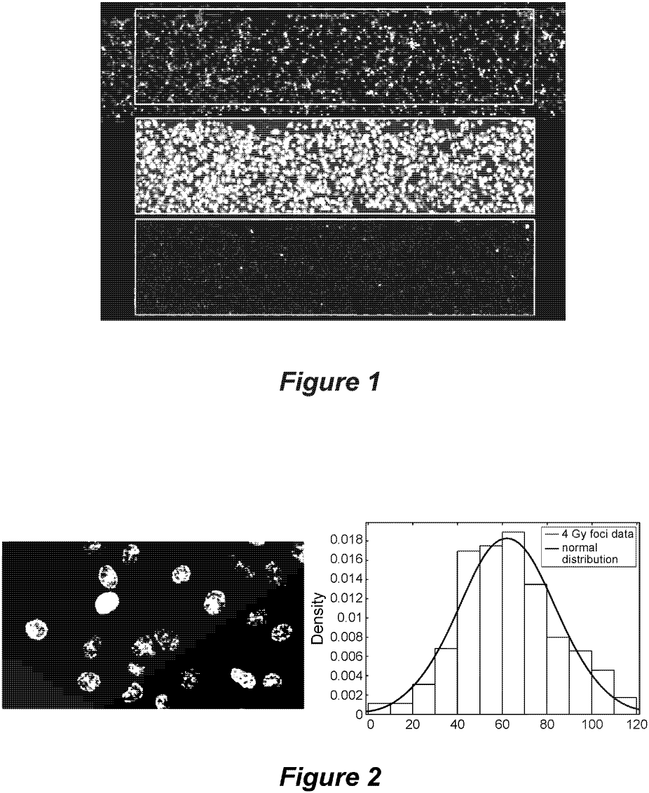

FIG. 1 shows an example of a cross correlative image set produced after irradiation with a clinical X-ray source. It consists of an image produced with X-ray Fluorescence (top) to image the nanoparticles.

The middle image in FIG. 1 is from confocal microscopy with a stain for DNA Double Strand Breaks (DSBs) that have not been repaired by the PC-3 cells within 1 hour after irradiation. The cells can then be defined and analysed with software for defining the cells and correlating information on the nanoparticle content in a cell and the number of DSBs in the same cell.

FIG. 2 shows an example of a zoomed in region of cells after irradiation with a 4 Gy dose from an Ir¹⁹² radioisotope source. Cell nuclei are dark as shown and DNA DSBs are lighter. The adjacent histogram shows the distribution of DNA DSBs in a cell population and a fit with a ‘normal’ distribution equation.

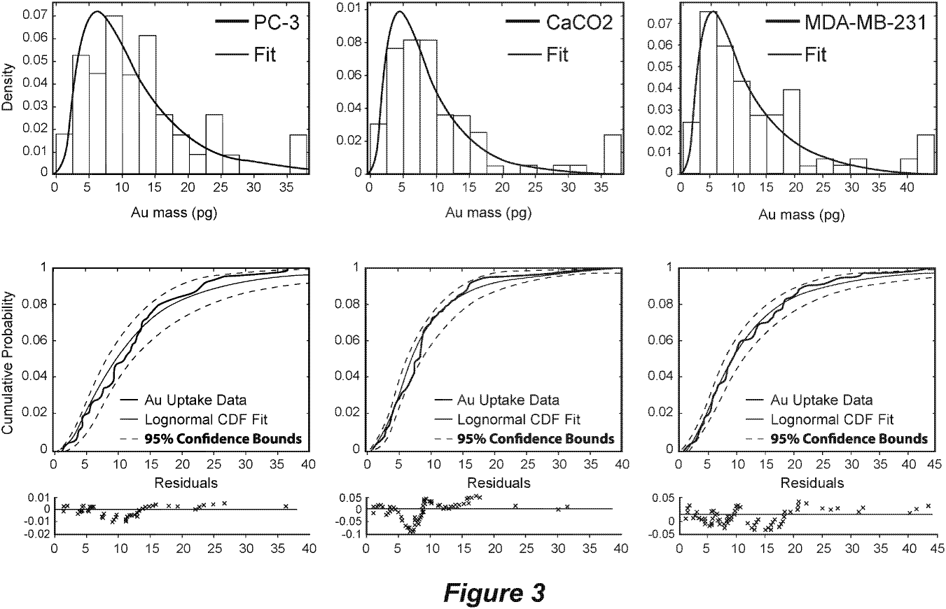

For the exact same cells data was produced from the XRF analysis that gives the quantity of nanoparticles in the same cells. This enables determination of probabilistic functions on nanoparticle uptake in addition to extracting information on correlations of the whole cell-population or sub-populations with biological attributes. Examples of data for the nanoparticle content are given for three different cancer cell lines (Prostate cancer, PC-3; Colorectal adenocarcinoma, CaCO2; and breast adenocarcinoma, MDA-MB-231) in FIG. 3.

The probability density function and the cumulative density functions can be described by:

$$P D F = \frac{1}{x σ \sqrt{2 π}} {e}^{- \frac{{\left(1 \text{n} \left(x\right) - μ\right)}^{2}}{2 {σ}^{2}}} \qquad \text{(1)}$$

$$C D F = \frac{1}{2} + \frac{1}{2} \text{erf} \left[\frac{ln \left(x\right) - μ}{\sqrt{2 {σ}^{2}}}\right] \qquad \text{(2)}$$

where µ = mean and σ = standard deviation.

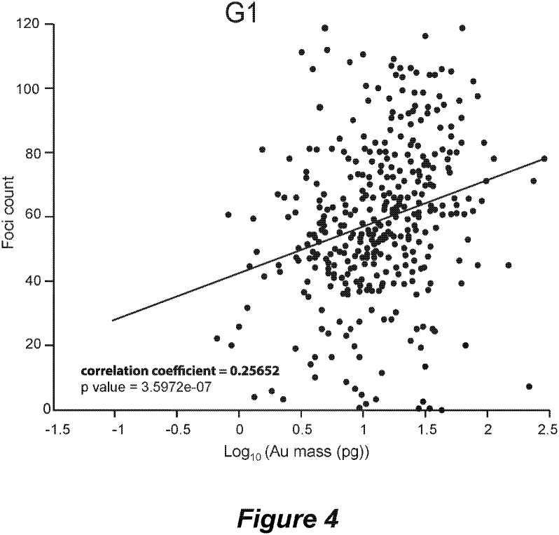

The data for each cell on number of DNA breaks (foci) and amount of gold nanoparticles can be plotted. An example for PC-3 cancer cells exposed to 4Gy from a clinical X-ray source is given in FIG. 4. There is a strong, positive and significant correlation.

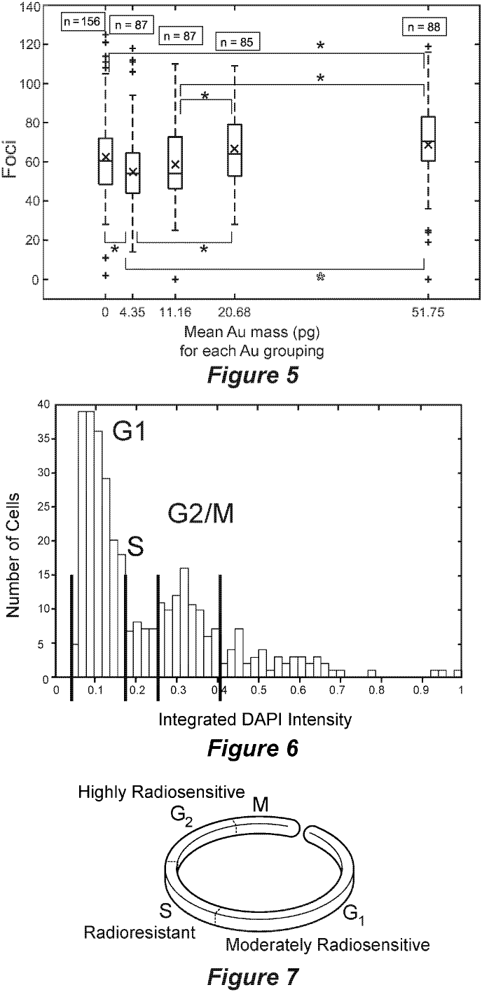

The data shown in FIG. 4 can be used for testing different quantities of nanoparticles in the sub-population of cells. After exposure to a radiation dose of 4Gy cells with below ~ 10pg of Au the nanoparticles instigate a cellular stress response which enhances the mechanisms for DNA repair (ie the number of foci are lower for the low Au content relative to the cells with no Au content). Above ~15 pg of Au the nanoparticles cause an impairment in the repair of DNA. In the plot shown in FIG. 5, the impairment in DNA repair is significant to the p<0.05 level at content greater than ~20 pg.

Due to this method having data on individual cells, we can further look at other markers of the cell, for example the amount of DNA in each cell (indicated by the stain DAPI). As the cell grows, the quantity of DNA increases through its growth phases. The cells can be divided into sub-populations based on their growth phase, as shown in FIG. 6.

The phases of cell growth have different sensitivity to radiation according to the DNA repair mechanisms that are available to the cell. The G1 phase is dominated by a DNA DSB repair mechanism call Non-Homologous End Joining (NHEJ). Through the S, G2 and M phases, DNA DSB repair is predominantly via Homologous Recombination, which are dependent on specific genes. These mechanisms have an important impact on cellular sensitivity to radiation repair (FIG. 7).

It is important to note that the cells in the S phase are radioresistant due to their ability to accurately repair DNA damage and this sub-population of cells correlate with poorer cancer prognosis and poorer therapy outcomes, ie these cells can be responsible for therapy failure.

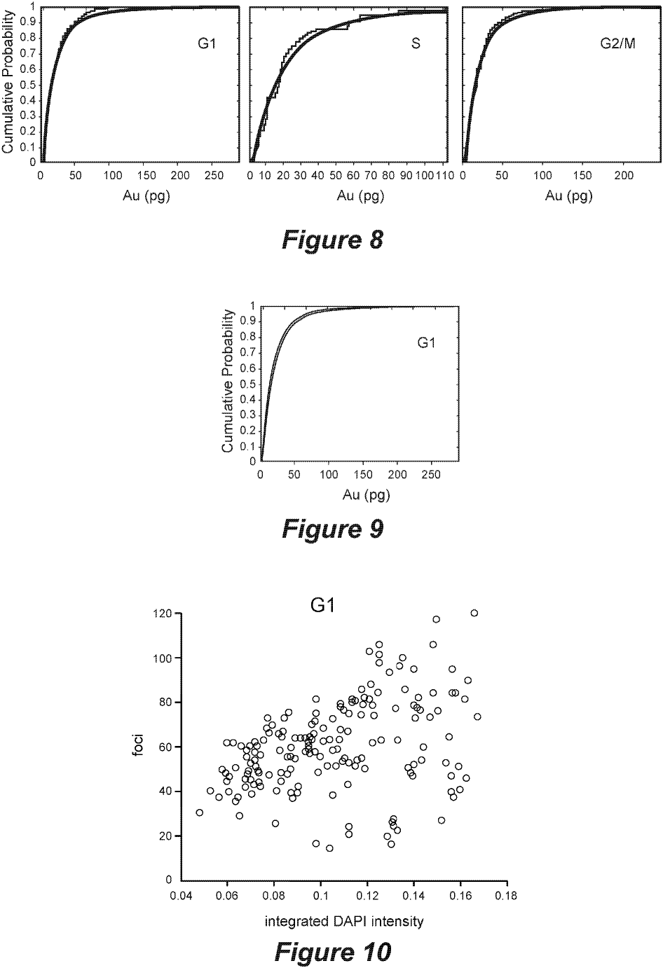

To show nanoparticles have specific effects on cells in different phases we needed to confirm the nanoparticle uptake probabilities are comparable for cells co-cultured with nanoparticles for a time of 2 hrs, or proportionally equivalent to ~10% of the cells’ doubling time. This is confirmed in the data shown in FIG. 8. In the overlay shown in FIG. 9 it can be seen that the three sub-populations are indistinguishable under these conditions with regard to the cumulative probability of nanoparticle uptake.

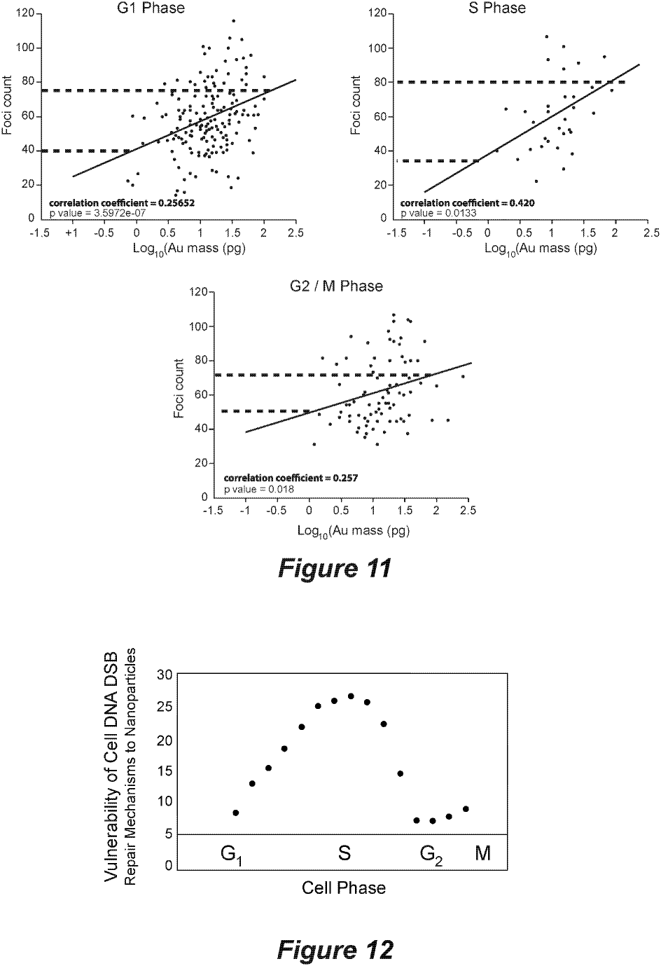

FIG. 10 shows that the sensitivity to radiation by way of ability to repair DNA varies within a specific growth phase, for example in the G1 phase. Furthermore, we have been able to show that the dependence on nanoparticle content on the cells’ DNA DSB repair mechanism through the growth phases varies. The data in FIG. 11 show the nanoparticles have least impact, by way of DNA DSB repair as a function of nanoparticle content (represented by the slope of the line fitting the data), on the most radiation sensitive cells (G2 and M phase). In other words, the repair of DNA DSBs decreases (i.e. more DNA DSBs are measured) as the content of nanoparticles increases and is most pronounced for the S phase cells.

By defining the slope of the line fitting these data as representing the vulnerability of cell DNA DSB repair mechanisms to be inhibited by nanoparticles, we can produce the data set shown in FIG. 12 showing the ability to impair DNA DSB repair varies through the cell cycle according the genetic state of the cells. Thus the cells in the S-phase, which correlate with cancer therapy failure, are the cells most prone to nanoparticle induced disruption of DNA DSB repair.

Each cell has an identical probability of experiencing DNA damage for an equivalent amount of nanoparticles, thus differences in the number of DNA DSBs between cells as a function of nanoparticle content in different phases are due to differences in the cells’ ability to repair the damage. The ability to repair the DNA DSBs is inversely correlated with the amount of nanoparticles in the cell. In this respect, nanoparticles are used to ‘prime’ the cell by impairing the cells repair mechanisms and renders the cell vulnerable to a subsequent therapy instigating DNA damage, such as X-rays, protons, neutron, other ions and chemotherapy drugs that act via causing DNA damage.

### Example 2 - Effect of Nanoparticles on Expression of Thymidylate Synthase

PC-3 cells were co-cultured with gold nanoparticles for 2 hours. Cells were then trypsinized and resuspended in phosphate buffer saline (PBS), washed several times by centrifugation and re-suspended. The supernatant was discarded and cells resuspended in 100 ul of primary antibody for thymidylate synthase (diluted in 1%BSA), incubated for 1 hour at 4° C. before washing again in PBS and centrifugation. The supernatant was discarded and the cell pellet resuspended in 100ul of secondary antibody (diluted in 1%BSA), incubated for 1 hour in dark at 4° C. and washing and centrifugation again. The supernatant was discarded and cell pellet resuspended with 40ul DAPI (1ug/ml), incubated for 15 min at room temperature in the dark before analysing cells on with flow cytometry.

Thymidylate synthase is a key enzyme in the synthesis of 2′-deoxythymidine-5′-monophosphate, an essential precursor for DNA biosynthesis. Thymidylate synthase therefore plays a crucial role in the early stages of DNA biosynthesis (Peters et al. 2002). Inhibition in synthesis of nucleotides necessary for cell growth is an important target for cancer treatment.

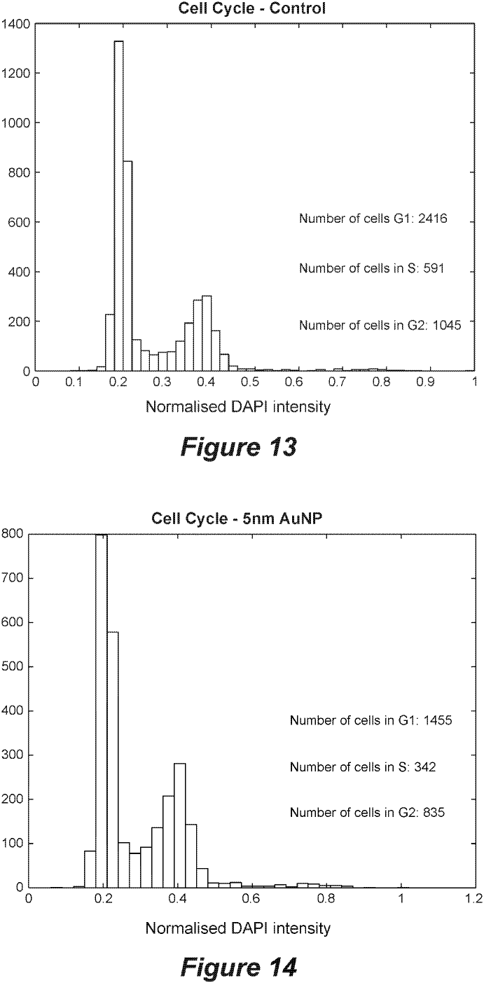

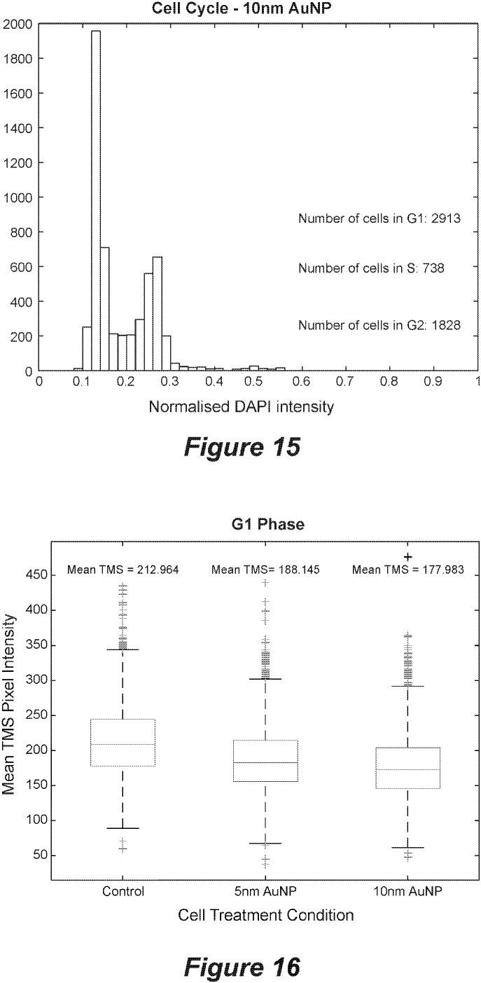

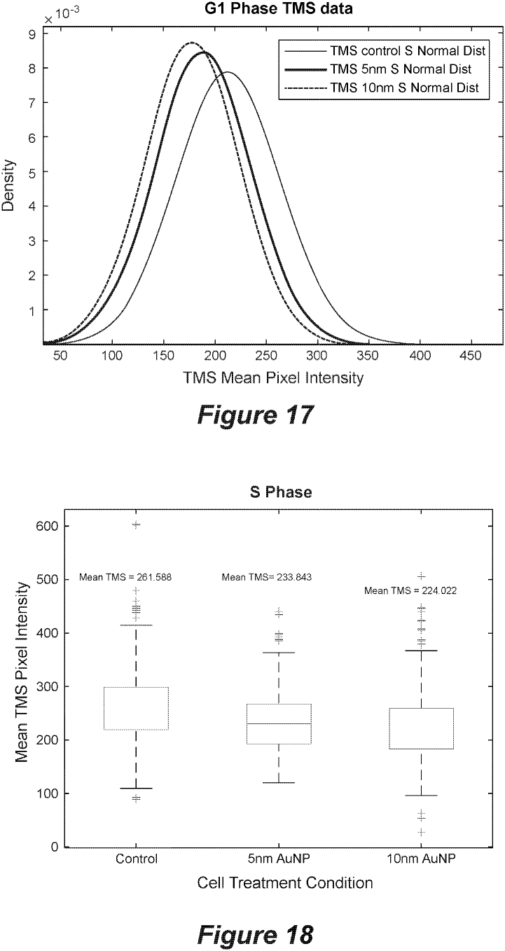

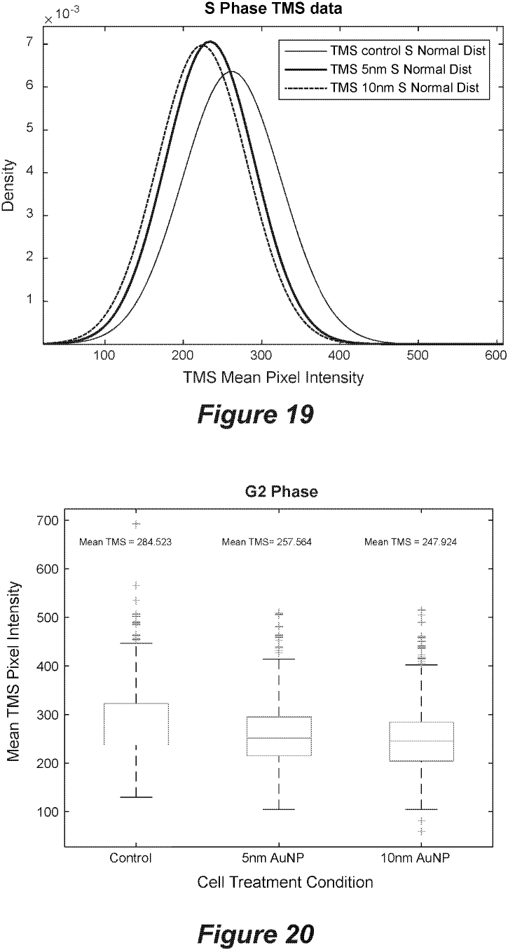

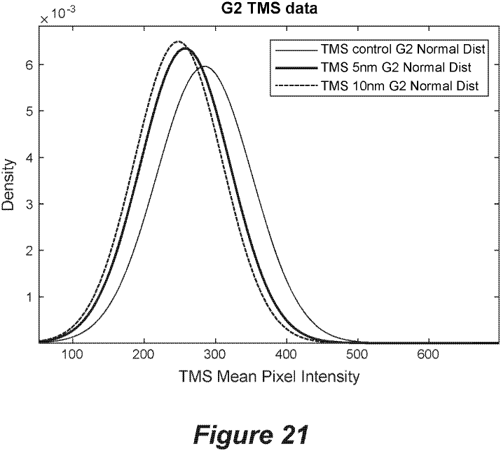

The data shown in FIGS. 13 to 21 shows that there is a statistical reduction in the expression of one of the genes involved in DNA repair. This shows that cells exposed to either 5 nm or 10 nm gold nanoparticles reduce the expression of thymidylate synthase, hence impairing the ability to the cells to recover after receiving subsequent DNA damage.

| | G1 | S | G2 |
|---|---|---|---|
| Control | 212.964 | 261.5883 | 284.5229 |
| 5 nm AuNP | 188.1449 | 233.8432 | 257.5638 |
| 10 AuNP | 177.9827 | 224.0216 | 247.9244 |

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

As used in this specification and the appended claims, the singular forms “a”, “an” and “the” include plural references unless the content clearly dictates otherwise.

As used herein, the term “about” when used in reference to a measurable value such as an amount of mass, dose, time, temperature, and the like, is meant to encompass variations of ±10%, ±5%, ±1%, ±0.5%, or even ±0.1% of the specified amount.

The reference to any prior art in this specification is not, and should not be taken as, an acknowledgement of any form of suggestion that such prior art forms part of the common general knowledge.

### REFERENCES

Hainfeld, J. F., D. N, Slatkin, et al. (2004). “The use of gold nanoparticles to enhance radiotherapy in mice.” Phys Med Biol 49(18): N309-15.

Kuo-Cheng Huang and Sheryl H. Ehrman (2007) “Synthesis of Iron Nanoparticles via Chemical Reduction with Palladium Ion Seeds” Langmuir, 23 (3), pp 1419-1426.

Liu T and Thierry B (2012) “A solution to the PEG dilemma: Efficient bioconjugation of large gold nanoparticles for biodiagnostic applications using mixed layers” Langmuir, 28 (44), 15634-15642.

Nath R, Anderson LL, Luxton G, Weaver KA, Williamson JF, Meigooni AS (1995) “Dosimetry of interstitial brachytherapy sources: Recommendations of the AAPM Radiation Therapy Committee Task Group No. 43” Medical Physics, 22 (2), 209-234.

Paterson D, de Jonge M.D., McKinlay J., Starritt A., Kusel M., Ryan C.G., Kirkham R., Moorhead G., Siddons D.P (2011) “The X-ray Fluorescence Microscopy Beamline at the Australian Synchrotron. AIP Conference 2011”; p. 219-222.

Peters GJ, Backus HH, Freemantle S, van Triest B, Codacci-Pisanelli G, van der Wilt CL, Smid K, Lunec J, Calvert AH, Marsh S, McLeod HL, Bloemena E, Meijer S, Jansen G, van Groeningen CJ, Pinedo HM (2002). “Induction of thymidylate synthase as a 5-fluorouracil resistance mechanism”. Biochim. Biophys. Acta. 1587 (2-3): 194-205.

Turkevich, J.; Stevenson, P. C.; Hillier, J. (1951). “A study of the nucleation and growth processes in the synthesis of colloidal gold”. Discuss. Faraday. Soc. 11: 55-75.

Turnbull T, Douglass M, Paterson D, Bezak E, Thierry B, Kempson I (2015) “Relating Intercellular Variability in Nanoparticle Uptake with Biological Consequence: A Quantitative X-ray Fluorescence Study for Radiosensitization of Cells” Analytical Chemistry, 87 (21), 10693-10697.

Turnbull T, Douglass M, Bezak E, Thierry B, Kempson I (2017) “An image processing application for quantitative cross-correlative microscopy for large cell-populations: a gold nanoparticle radiosensitisation study” Powder Diffraction, 1-5.

1. A method of increasing the amount of strand breaks in DNA in a cell, the method comprising:
exposing the cell to an effective amount of a nanoparticle composition comprising biocompatible nanoparticles under conditions in which at least some of the nanoparticles are localised in the cell to form a nanoparticle-laden cell and the localised nanoparticles alter one or more cell regulatory mechanisms in the nanoparticle-laden cells or other cells.

2. The method of claim 1, further comprising exposing the nanoparticle-laden cell to a chemotherapeutic agent or ionizing radiotherapy concurrently with or after the nanoparticles have altered the one or more cell regulatory mechanisms in the nanoparticle-laden cells or other cells.

3. A method of inducing cancer cell death, the method comprising:
exposing cancer cells to be treated to an effective amount of a nanoparticle composition comprising biocompatible nanoparticles under conditions in which at least some of the nanoparticles are localised in the cancer cells to form nanoparticle-laden cancer cells and the localised nanoparticles alter one or more cell regulatory mechanisms in the nanoparticle-laden cancer cells or other cells; and
exposing the nanoparticle-laden cancer cells to a chemotherapeutic agent or ionizing radiotherapy concurrently with or after the nanoparticles have altered the one or more cell regulatory mechanisms in the nanoparticle-laden cancer cells or other cells under conditions to cause cancer cell death.

4. The method of claim 3, wherein the cancer is selected from the group consisting of breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms’ tumour, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukaemia, acute lymphocytic leukaemia, chronic lymphocytic leukaemia, acute myelogenous leukaemia, chronic myelogenous leukaemia, chronic granulocytic leukaemia, acute granulocytic leukaemia, hairy cell leukaemia, neuroblastoma, rhabdomyosarcoma, Kaposi’s sarcoma, polycythemia vera, essential thrombocytosis, Hodgkin’s disease, non-Hodgkin’s lymphoma, soft-tissue sarcoma, osteogenic sarcoma, primary macroglobulinemia, and retinoblastoma.

5. A chemotherapeutic or radiotherapeutic treatment method comprising:
administering to a subject in need of chemotherapeutic or radiotherapeutic treatment an effective amount of a nanoparticle composition comprising biocompatible nanoparticles; and
administering one or more doses of a chemotherapeutic agent or ionizing radiotherapy to the subject either concurrently with or after administration of the nanoparticle composition;
wherein the nanoparticle composition is administered under conditions in which the nanoparticles alter one or more cell regulatory mechanisms in cells in which the nanoparticles are localised, or other cells, and one or more doses of a chemotherapeutic agent or ionizing radiotherapy are administered to the subject either concurrently with or after the nanoparticles have altered the one or more cell regulatory mechanisms in cells in which the nanoparticles are localised or other cells.
